(19) United States
(12) Patent Application Publication
(10) Pub. No.: US 2021/0147413 A1
(43) Pub. Date: May 20, 2021

(54) ISOTHERMAL REACTIVE CRYSTALLISATION PROCESS FOR THE PREPARATION OF A CRYSTALLINE FORM OF PIMODIVIR HYDROCHLORIDE HEMIHYDRATE

(71) Applicant: Janssen Pharmaceuticals, Inc., Titusville, NJ (US)

(72) Inventors: Alain Rudi G. COLLAS, Aartselaar (BE); Tim Joeri GAERDEN, Turnhout (BE); Micha Wilhelmina Jozefus Maria PEETERS, Baarle-Nassau (NL)

(21) Appl. No.: 17/045,150

(22) PCT Filed: Feb. 6, 2019
(86) PCT No.: PCT/IB2019/050932

§ 371 Date: Oct. 2, 2020

Related U.S. Application Data
(60) Provisional application No. 62/654,102, filed on Apr. 6, 2018.

(30) Foreign Application Priority Data
Apr. 6, 2018 (EP) 18166075.4

(51) Int. Cl. C07D 471/04

(52) U.S. Cl. CPC C07D 471/04; C07B 2200/13

(57) ABSTRACT

The present invention relates to an isothermal reactive crystallisation procedure to obtain the HCl salt of (2S,3S)-3-{[5-fluoro-2-(5-fluoro-1H-pyrrolo[2,3-b]pyridin-3-yl)pyrimidin-4-yl]amino}-bicyclo[2.2.2] octane-2-carboxylic acid hemihydrate in crystalline form using a solvent system comprising a mixture of water and one or more organic solvents.

This invention was made with Government support under contract number HHSO100201500014C awarded by the Office of the Assistant Secretary for Preparedness and Response, Biomedical Advanced Research and Development Authority. The Government has certain rights in the invention.

The present invention relates to an isothermal reactive crystallisation procedure to obtain the HCl salt of (2S,3S)-3-{[5-fluoro-2-(5-fluoro-1H-pyrrolo[2,3-b]pyridin-3-yl)pyrimidin-4-yl]amino}bicyclo[2.2.2]octane-2-carboxylic acid hemihydrate in crystalline form using a solvent system comprising a mixture of water and one or more organic solvents.

Compound (1) i.e. (2S,3S)-3-{[5-fluoro-2-(5-fluoro-1H-pyrrolo[2,3-b]pyridin-3-yl)-pyrimidin-4-yl]amino}bicyclo[2.2.2]octane-2-carboxylic acid, which can represented by the structural formula:

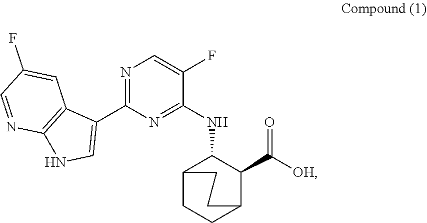 [SMILES: O=C(O)[C@H]1C2CCC(CC2)[C@@H]1Nc1nc(-c2c[nH]c3ncc(F)cc23)ncc1F]

and pharmaceutically acceptable salts thereof can inhibit the replication of influenza viruses and has been described in WO-2010/148197. This compound is also known under its international Nonproprietary Names (INN) as pimodivir.

The HCl salt of the hemihydrate form of Compound (1) has been disclosed in WO-2015/073476. Also disclosed therein are methods for preparing the hydrochloric acid salt of Compound (1) hemihydrate.

Compound (1) can exist in or form different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition that are distinct in their crystalline structures and typically have different physicochemical properties. Generally, different polymorphs can be characterized by analytical methods such as X-ray powder diffraction (XRPD) pattern, thermogravimetric analysis (TGA), and differential scanning calorimetry (DSC), or by its melting point, or other techniques known in the art. The HCl salt of the hemihydrate form of Compound (1) as prepared according to the crystallisation procedure of the present invention is identical to the polymorphic Form A of HCl salt of Compound (1) hemihydrate as disclosed in WO-2015/073476.

As used herein, “Compound (1)” means the free base form of Compound (1). Accordingly, “HCl salt of Compound (1)” means a 1 to 1 HCl salt of the free base compound. The HCl salt of Compound (1) hemihydrate is the HCl salt of Compound (1) that includes water as a solvate in a half equivalent per Compound (1).

Crystal engineering is of importance in the production of Active Pharmaceutical Ingredients (APIs). During crystallisation, many physicochemical characteristics of the API or drug substance are defined, including crystal polymorph, shape, size, particle size distribution, chemical purity and stability. These characteristics influence the stirrability, residual solvent level, drying time, agglomeration, fragmentation and attrition during the isolation process, which in turn affects the drug product manufacturing by determining particle flow, compressibility, solubility, dissolution rate and bioavailability. The specifications towards the physical properties of the API, driven by the drug product manufacturing, are very narrow concerning particle size distribution, specific surface area, bulk density, triboelectrification and flowability.

WO-2015/073476 discloses in paragraph 0053 on page 11 and in Example 3 a traditional reactive crystallisation process for preparing the HCl salt of Compound (1) hemihydrate using heating a solution followed by cooling to induce crystallisation in a solvent system comprising of water and one or more organic solvents having a water activity of 0.05 to 0.85. This process is characterized by preparing a solution, slurry or suspension of the 2-methyl tetrahydrofuran (2-MeTHF) solvate of Compound (1) in a mixture of water and an organic solvent such as acetone, n-propanol, isopropanol, acetic acid, or mixtures thereof, and subsequently heating said solution, slurry or suspension and treating it with HCl, followed by cooling to 0° C. and isolating the formed crystals by filtration.

It has been observed that the HCl salt of the hemihydrate form of Compound (1) prepared according to the above described procedure of WO-2015/073476 yields crystalline HCl salt of Compound (1) hemihydrate having a very broad particle size distribution that form agglomerates as can be seen in FIGS. 1 and 2.

It has now been found that crystalline HCl salt of the hemihydrate form of Compound (1) can alternatively be prepared using an isothermal procedure (i.e. no cooling to obtain crystallisation) wherein Compound (1), or a solvate thereof, is dissolved in a mixture of water and one or more organic solvents followed by addition of aqueous HCl to this solution giving an in-situ formation of a seed bed, followed by further addition of Compound (1), or a solvate thereof, whereby the temperature is kept constant (i.e. isothermally) during the whole process. The crystalline HCl salt of Compound (1) in its hemihydrate form prepared according to this method has a narrow particle size distribution and a well-defined morphology as can be seen in FIGS. 3 and 4.

The narrow particle size distribution and well-defined morphology of the crystalline HCl salt of the hemihydrate form of Compound (1) prepared according to the present invention has the following advantages:
less subjective to scale-up effects, more robust process
easier to wash and dry (smaller Loss on Drying) preventing unwanted agglomeration
less cohesiveness and tendency to electrostatically charge due to larger particle size and decreased specific surface area
increased bulk density
better powder flowability

In an embodiment the present invention relates to a process for preparing crystalline HCl salt of the hemihydrate form of Compound (1) comprising the consecutive steps of
a) dissolving Compound (1), or a solvate thereof, in a solvent system comprising a mixture of water and one or more organic solvents and having a water activity of 0.05 to 0.85;
b) heating the mixture of step a) until all of Compound (1), or a solvate thereof, is dissolved;
c) gradually adding an amount of aqueous HCl solution to the mixture of step b);
d) keeping the mixture of step c) for a prolonged period;
e) gradually adding further Compound (1), or a solvate thereof;
f) cooling the mixture of step e) to room temperature; and
g) isolating the crystals of HCl salt of Compound (1) hemihydrate thus formed;
   characterized in that the steps b) to e) are performed isothermally (i.e. at the same constant temperature) which can be any specific temperature ranging from 20° C. to the reflux temperature of the solvent system.

Examples of solvates of Compound (1) include solvates of 2-MeTHF, N,N-dimethylacetamide, N,N-dimethylformamide, methanol, xylene, acetone, 2-butanol, methyl acetate, 1-pentanol, 2-propanol, tetrahydrofuran, methyl tetrahydrofuran, 1,4-dioxane, 1-pentanol, 2-methyl-1-propanol, methylethyl ketone, 3-methyl-1-butanol, heptane, ethyl formate, 1-butanol, acetic acid, and ethylene glycol. In a specific embodiment, solvates of 2-MeTHF (i.e. Compound (1).1(2-MeTHF)) are employed.

The solvent systems suitable for the preparation of the HCl salt of Compound (1) hemihydrate are comprised of a large variety of combinations of water and one or more organic solvents. Suitable organic solvents include Class II or Class III organic solvents listed in the International Conference on Harmonization Guidelines. Specific examples of suitable Class II organic solvents include chlorobenzene, cyclohexane, 1,2-dichloroethene, dichloromethane, 1,2-dimethoxyethane, N,N-dimethylacetamide, N,N-dimethylformamide, 1,4-dioxane, 2-ethoxyethanol, formamide, hexane, 2-methoxyethanol, methyl butyl ketone, methylcyclohexane, N-methylpyrrolidone, nitromethane, pyridine, sulfolane, tetrahydrofuran (THF), tetralin, toluene, 1,1,2-trichloroethene and xylene. Specific examples of suitable Class III organic solvents include: acetic acid, acetone, anisole, 1-butanol, 2-butanol, butyl acetate, tert-butylmethyl ether, cumene, heptane, isobutyl acetate, isopropyl acetate, methyl acetate, 2-methyl-1-butanol, methylethyl ketone, methylisobutyl ketone, 2-methyl-1-propanol, ethyl acetate, ethyl ether, ethyl formate, pentane, 1-pentanol, 1-propanol, 2-propanol and propyl acetate. In one specific embodiment, the organic solvents of the solvent system are selected from the group consisting of chlorobenzene, cyclohexane, 1,2-dichloroethane, dichloromethane, 1,2-dimethoxyethane, hexane, 2-methoxyethanol, methyl butyl ketone, methylcyclohexane, nitromethane, tetralin, xylene, toluene, 1,1,2-trichloroethane, acetone, anisole, 1-butanol, 2-butanol, butyl acetate, tert-butylmethyl ether, cumene, ethanol, ethyl acetate, ethyl ether, ethyl formate, heptane, isobutyl acetate, isopropyl acetate, methyl acetate, 3-methyl-1-butanol, methylethyl ketone, 2-methyl-1-propanol, pentane, 1-propanol, 1-pentanol, 2-propanol, propyl acetate, tetrahydrofuran, and methyl tetrahydrofuran. In another specific embodiment, the organic solvents of the solvent system are selected from the group consisting of 2-ethoxyethanol, ethyleneglycol, methanol, 2-methoxyethanol, 1-butanol, 2-butanol, 2-methyl-1-butanol, 2-methyl-1-propanol, ethanol, 1-pentanol, 1-propanol, 2-propanol, methyl butyl ketone, acetone, methyl ethyl ketone, methyl isobutyl ketone, butyl acetate, isobutyl acetate, isopropyl acetate, methyl acetate, ethyl acetate, propyl acetate, pyridine, toluene, and xylene. In yet another embodiment, the organic solvents are selected from the group consisting of acetone, ethanol, dichloromethane, methyl ethyl ketone, 2-methyl-1-butanol and ethyl acetate. In yet another embodiment, the solvent system consists of water and acetone.

The solvent system comprising of a mixture of water and one or more organic solvents has a water activity of 0.05 to 0.85. In an embodiment where the solvent system is a mixture of water and acetone, the value of the water activity is 0.2 to 0.8, in particular from 0.4 to 0.6.

The term ‘water activity’ (a_{w}) is used herein as known in the art and means a measure of the energy status of water in a solvent system. It is defined as the vapor pressure of a liquid divided by that of pure water at the same temperature. Specifically, it is defined as a_{w}=p/pₒ, where p is the vapor pressure of water in the liquid, and pₒ is the vapor pressure of pure water at the same temperature, or as a_{w}=l_{w}×x_{w}, where l_{w} is the activity coefficient of water and x_{w} is the mole fraction of water in the aqueous fraction. For example, pure water has a water activity value of 1.0. Water activity values can typically be obtained by either a capacitance hygrometer or a dew point hygrometer. Various types of water activity measuring instruments are also commercially available. Alternatively, water activity values of mixtures of two or more solvents can be calculated based on the amounts of the solvents and the known water activity values of the solvents.

The crystallisation process steps b) to e) are performed isothermally whereby the temperature is kept constant at any specific temperature ranging from 20° C. to the reflux temperature of the solvent system. Typically, the isothermal temperature for steps b) to e) is any temperature from 40° C. to 80° C. In an embodiment wherein the solvent systems consists of a mixture of water and acetone, the isothermal temperature is any temperature from 20° C. to 56° C., or from 40° C. to 56° C., in particular the isothermal temperature is 50° C.

The amount of Compound (1), or solvate thereof, used in step a) can range from 5% to 30% of the total amount of Compound (1), or solvate thereof, used in step a) and e) together. In an embodiment, the amount of Compound (1), or solvate thereof, used in step a) can range from 10% to 20% of the total amount of Compound (1), or solvate thereof, used in step a) and e) together.

The amount of HCl in the aqueous HCl solution added in step c) ranges from 1.0 equivalent to 2.0 equivalent compared to the total amount of Compound (1) used in step b) and e) together, or a solvate thereof. In an embodiment the amount of HCl ranges from 1.0 to 1.30 equivalents, or from 1.10 to 1.20 equivalents. In another embodiment, the amount of HCl is 1.15 equivalents.

The aqueous HCl solution added in step c) is gradually added to the mixture obtained in step b) over a period of 5 minutes to 120 minutes. In an embodiment, the period ranges from 45 to 75 minutes. In another embodiment, the period is 60 minutes.

In step d) the mixture is maintained at the same temperature as in step b) for a prolonged period. In an embodiment this prolonged period is a period of 6 to 36 hours, preferably of 7 to 25 hours, more preferably of 7 to 15 hours. In another embodiment this period ranges from 7 to 9 hours. In yet another embodiment the period is 8 hours. During this period, also called the aging period since it allows for Ostwald ripening to occur, a seed bed of crystalline HCl salt of Compound (1) hemihydrate is formed.

In step e) a further amount of Compound (1), or a solvate thereof, is added to the mixture of step d). In a preferred embodiment, the amount of compound (1) added is of from 0.4 to 0.99 eq., preferably from 0.6 to 0.97 eq., more preferably from 0.7 to 0.9 eq, most preferably from 0.57 to 0.86 eq. Compound (1), or a solvate thereof, can be added in solid form or dissolved in a solvent system comprising a mixture of water and one or more organic solvents wherein said solvent system has a water activity of 0.05 to 0.85. This solvent system can be the same or different to the solvent system used in step a). In practice the solvent system in step e) is not the same as in step a) and has a higher water activity than used in step a). The water activity ratio of the solvent system in step e) to the solvent system in step a) is 1:1, preferably 1:1.4, more preferably 1:1.8, even more preferably 1:2, most preferably 1:3.

In step e) a further solution comprising Compound (1), or a solvate thereof, is gradually added over a period of 1 hours to 12 hours. In an embodiment, the period ranges from 7 to 9 hours. In another embodiment, the period is 8 hours.

In step f) the crystallisation mixture is allowed to cool to room temperature before the formed crystals are isolated in step g). The cooling may be done by natural cooling or according to a specific temperature cooling profile. For instance, the temperature cooling profile may be a linear profile, e.g. 0.1° C./minute, 0.3° C./minute, 0.5° C./minute, 0.75° C./minute, 1° C./minute, 2° C./minute or any other value.

The isolation of the crystalline HCl salt of the hemihydrate form of Compound (1) in step g) can be carried out by any conventional means, such as by filtration or centrifugation.

Particle size analysis of the crystalline HCl salt of Compound (1) hemihydrate in suspension during the crystallization process can be performed with in-line process analytical technology techniques such as focused beam reflectance measurement (FBRM) using e.g. the Lasentec® products from Mettler-Toledo. Alternatively, samples can be taken at different times during the crystallisation procedure and analysed using laser diffraction techniques with suitable equipment such as e.g. a Malvern Mastersizer 2000 laser diffractometer (Malvern, UK).

In a further embodiment the isothermal reactive crystallization process of the present invention can also be used for crystallizing any other drug substance. The term ‘drug substance’ is an ‘active ingredient’ which is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. In a preferred embodiment, the active ingredient is a salt.

### EXAMPLE 1

Reactor 1 is charged with 0.14 equivalents of the 2-methyltetrahydrofuran solvate of (2S,3S)-3-{[5-fluoro-2-(5-fluoro-1H-pyrrolo[2,3-b]pyridin-3-yl) pyrimidin-4-yl]amino}bicyclo[2.2.2]octane-2-carboxylic acid, 2.80 L/mole acetone and 0.093 L/mole water. The mixture is stirred and heated to 50° C. over 1 hour. The resulting solution is transferred to Reactor 2 over a filter in order to remove any residual insoluble matter. The solution in Reactor 2 is maintained at 50° C. while 1.15 equivalents (minimum 34 w %) HCl (aqueous) is dosed over 1 hour. The lines are rinsed with 0.3 L/mole acetone to remove any remaining HCl solution. The in-situ precipitated seed bed of HCl salt of (2S,3S)-3-{[5-fluoro-2-(5-fluoro-1H-pyrrolo[2,3-b]pyridin-3-yl)pyrimidin-4-yl]amino}bicyclo[2.2.2] octane-2-carboxylic acid hemihydrate is aged for 8 hours. In the meanwhile, 0.86 equivalents of the 2-methyltetrahydrofuran solvate of (2S,3S)-3-{[5-fluoro-2-(5-fluoro-1H-pyrrolo[2,3-b]pyridin-3-yl)pyrimidin-4-yl]amino}bicyclo[2.2.2]octane-2-carboxylic acid is dissolved in a mixture of 4.81 L/mole acetone and 0.42 L/mole water at 50° C. in Reactor 1. The homogeneous content of Reactor 1 is dosed over a filter to the seed bed in Reactor 2 over 8 hours at 50° C. After completion of dosing of the base solution, the suspension is cooled to 20° C. to isolate the product by filtration. The filter cake is immediately washed with 1 L/mole acetone to remove the mother liquor. The HCl salt of (2S,3S)-3-{[5-fluoro-2-(5-fluoro-1H-pyrrolo[2,3-b]pyridin-3-yl)pyrimidin-4-yl]amino}bicyclo[2.2.2]octane-2-carboxylic acid hemihydrate product is dried at 40° C./200 mbar.

### DESCRIPTION OF THE DRAWINGS

FIG. 1: particle size distribution of HCl salt of Compound (1) hemihydrate prepared according to the procedure described in example 3, first line of table 1 of WO-2015/073476 (SEM at scale 200 μm and 50 μm)

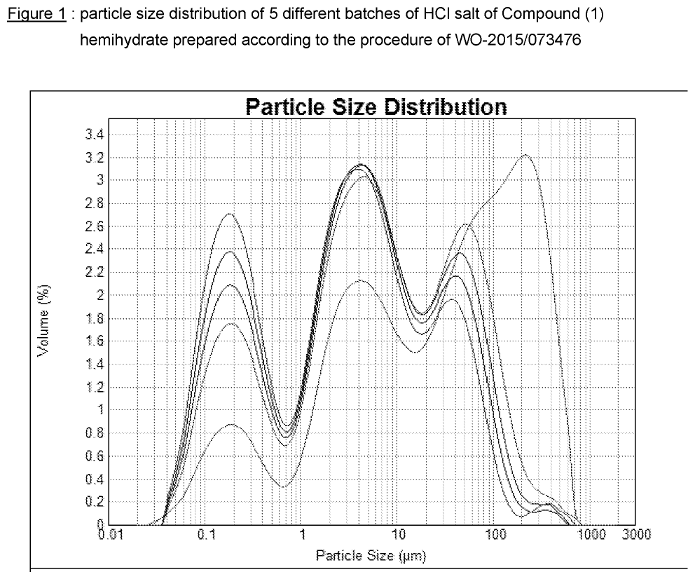

FIG. 2: morphology of crystals of HCl salt of Compound (1) hemihydrate prepared according to the procedure described in example 3, first line of table 1 of WO-2015/073476

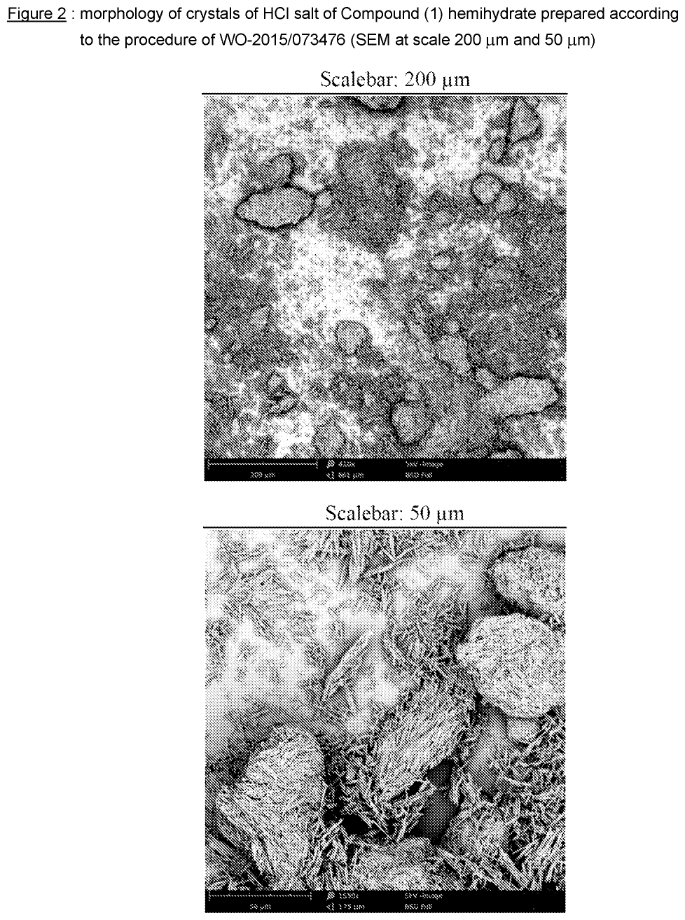

FIG. 3: particle size distribution of HCl salt of Compound (1) hemihydrate prepared according to the present invention

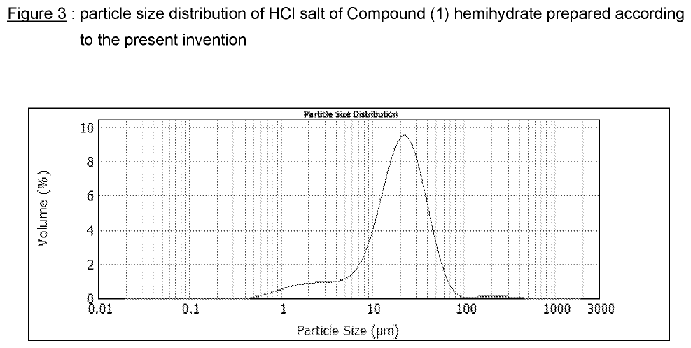

FIG. 4: morphology of crystals of HCl salt of Compound (1) hemihydrate prepared according to the procedure of the present invention (SEM at scale 200 μm and 50 μm)

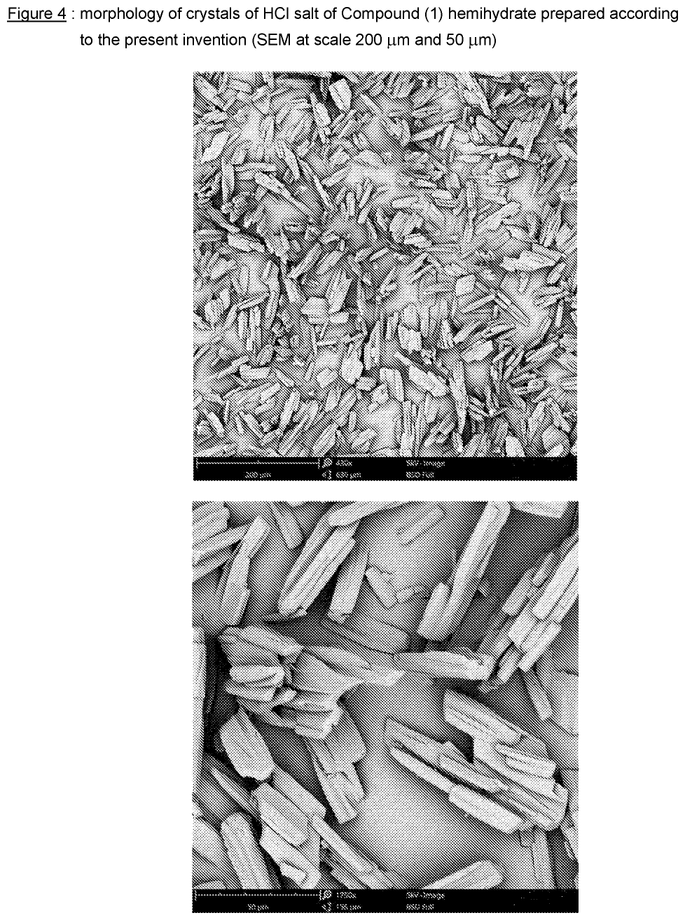

1. A process for preparing crystalline HCl salt of the hemihydrate form of Compound (1) 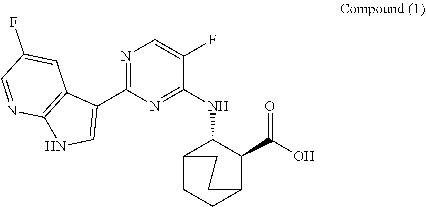 [SMILES: O=C(O)[C@H]1C2CCC(CC2)[C@@H]1Nc1nc(-c2c[nH]c3ncc(F)cc23)ncc1F]
comprising the consecutive steps of
a) dissolving Compound (1), or a solvate thereof, in a solvent system comprising a mixture of water and one or more organic solvents and having a water activity of 0.05 to 0.85;
b) heating the mixture of step a) until all of Compound (1), or a solvate thereof, is dissolved;
c) gradually adding an amount of aqueous HCl solution to the mixture of step b);
d) keeping the mixture of step c) for a prolonged period of 6 to 36 hours;
e) gradually adding further Compound (1), or a solvate thereof;
f) cooling the mixture of step e) to room temperature; and
g) isolating the crystals of HCl salt of Compound (1) hemihydrate thus formed;
characterized in that the steps b) to e) are performed isothermally which can be any specific temperature ranging from 20° C. to the reflux temperature of the solvent system.

2. The process according to claim 1 wherein the one or more organic solvents in the solvent system are selected from Class II or Class III organic solvents.

3. The process according to claim 2 wherein the Class II organic solvents are selected from chlorobenzene, cyclohexane, 1,2-dichloroethene, dichloromethane, 1,2-dimethoxyethane, N,N-dimethylacetamide, N,N-dimethylformamide, 1,4-dioxane, 2-ethoxyethanol, formamide, hexane, 2-methoxyethanol, methyl butyl ketone, methylcyclohexane, N-methylpyrrolidone, nitromethane, pyridine, sulfolane, tetrahydrofuran (THF), tetralin, toluene, 1,1,2-trichloroethene and xylene.

4. The process according to claim 2 wherein the Class III organic solvents are selected from acetic acid, acetone, anisole, 1-butanol, 2-butanol, butyl acetate, tert-butylmethyl ether, cumene, heptane, isobutyl acetate, isopropyl acetate, methyl acetate, 2-methyl-1-butanol, methylethyl ketone, methylisobutyl ketone, 2-methyl-1-propanol, ethyl acetate, ethyl ether, ethyl formate, pentane, 1-pentanol, 1-propanol, 2-propanol and propyl acetate. In one specific embodiment, the organic solvents of the solvent system are selected from the group consisting of chlorobenzene, cyclohexane, 1,2-dichloroethane, dichloromethane, 1,2-dimethoxyethane, hexane, 2-methoxyethanol, methyl butyl ketone, methylcyclohexane, nitromethane, tetralin, xylene, toluene, 1,1,2-trichloroethane, acetone, anisole, 1-butanol, 2-butanol, butyl acetate, tert-butylmethyl ether, cumene, ethanol, ethyl acetate, ethyl ether, ethyl formate, heptane, isobutyl acetate, isopropyl acetate, methyl acetate, 2-methyl-1-butanol, methyl ethyl ketone, 2-methyl-1-propanol, pentane, 1-propanol, 1-pentanol, 2-propanol, propyl acetate, tetrahydrofuran, and methyl tetrahydrofuran.

5. The process according to claim 1 wherein the isothermal temperature for steps b) to e) ranges from 40° C. to 80° C.

6. The process according to claim 1 wherein the solvent system consists of water and acetone.

7. The process according to claim 6 wherein solvent system has a water activity from 0.2 to 0.8, in particular from 0.4 to 0.6.

8. The process according to claim 7 wherein Compound (1) in step a) is in the form of a 2-methyltetrahydrofuran (2-MeTHF) solvate.

9. The process according to claim 8 wherein the isothermal temperature for steps b) to e) ranges from 20° C. to 56° C., or from 40° C. to 56° C., in particular the isothermal temperature is 50° C.

10. The process according to any one of claims 1 to 9 wherein the amount of HCl in the aqueous HCl solution added in step c) ranges from 1.0 equivalent to 2.0 equivalent compared to the total amount of Compound (1), or a solvate thereof, used in steps b) and e) together.

11. The process according to claim 10 wherein the amount of HCl ranges from 1.0 to 1.30 equivalents, or from 1.10 to 1.20 equivalents.

12. The process according to claim 10 or claim 11 wherein the aqueous HCl solution is gradually added over a period of 5 minutes to 120 minutes, in particular over a period from 45 to 75 minutes.

13. The process according to any one of the preceding claims wherein the mixture in step d) is maintained for a period of 7 to 9 hours.

14. The process according to any one of the preceding claims wherein in step e) further Compound (1), or a solvate thereof, is added gradually.

15. The process according to claim 14 wherein Compound (1), or a solvate thereof, is gradually added over a period of 1 to 12 hours, in particular over a period of 7 to 9 hours.

16. The process according to claim 14 wherein Compound (1), or a solvate thereof, is gradually added in solid form.

17. The process according to claim 14 wherein Compound (1), or a solvate thereof, is gradually added dissolved in a solvent system comprising a mixture of water and one or more organic solvents wherein said solvent system has a water activity of 0.05 to 0.85.

18. The process according to claim 17 wherein the solvent system is as defined in any one of claims 2 to 6.

19. The process according to any one of the preceding claims wherein the crystalline HCl salt of the hemihydrate form of Compound (1) is isolated in step g) by filtration.
